(19) United States
(12) Patent Application Publication
(10) Pub. No.: US 2004/0266795 A1
(43) Pub. Date: Dec. 30, 2004

(54) Process for the preparation of famciclovir

(76) Inventors: Genny Shamai, Kiryat Yam (IL); Shlomo Antebi, Haifa (IL); David Ioffe, Ramot Sapir (IL); Ben-Zion Dolitzky, Petach Tiqva (IL); Batia Kauffmann, Carmiel (IL)

Correspondence Address: KENYON & KENYON, ONE BROADWAY, NEW YORK, NY, 10004, US

(21) Appl. No.: 10/836,028

(22) Filed: Apr. 30, 2004

Related U.S. Application Data
(60) Provisional application No. 60/488,268, filed on Jul. 16, 2003.
(60) Provisional application No. 60/466,705, filed on Apr. 30, 2003.

(51) Int. Cl. A61K 31/52; C07D 473/00

(52) U.S. Cl. 514/263.1; 544/264

(57) ABSTRACT

The invention provides processes for making famciclovir with low levels of undesirable by-products. The present invention discloses a process comprises reacting a compound of formula I (acetic acid 2-acetoxymethyl-4-(5-amino-7-chloro-imidazo[4,5-b]pyridin-3-yl)-butyl ester) in the presence of a palladium on charcoal catalyst in a C₁-C₆ alkyl acetate and ammonium formate. The present invention further discloses a process comprises reacting a compound of formula I (acetic acid 2-acetoxymethyl-4-(5-amino-7-chloro-imidazo[4,5-b]pyridin-3-yl)-butyl ester) in the presence of a palladium on charcoal catalyst in a mixture of a C₁-C₆ alkyl acetate, a C₁-C₄ alcohol and ammonium formate.

### CROSS-REFERENCE TO RELATED APPLICATIONS

This application claims the benefit of the U.S. Provisional Application Ser. No. 60/466,705 filed Apr. 30, 2003 and 60/488,268 filed Jul. 16, 2003, the disclosures of which are incorporated by reference in their entireties herein.

### FIELD OF THE INVENTION

The invention relates to processes for preparing famciclovir.

### BACKGROUND OF THE INVENTION

Famciclovir was developed as an orally administered antiviral drug by SmithKline Beecham and is available as Famvir®. Famvir® is indicated for the treatment of acute herpes zoster (shingles). It is also indicated for treatment or suppression of recurrent genital herpes in immunocompetent patients and for treatment of recurrent mucocutaneous herpes simplex infections in HIV infected patients. Famciclovir has the following chemical formula:

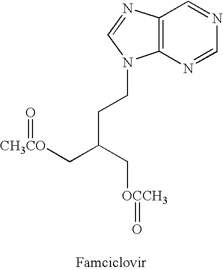

### Famciclovir

The chemical name for famciclovir is 2-[2-(2-amino-9H-purin-9-yl)ethyl]-1,3-propane diacetate. It is reported to be a white to pale yellow solid that is freely soluble in acetone and methanol, but sparingly soluble in ethanol and isopropanol. In the anhydrous form, at 25° C., it is reported to be freely soluble in water (>25% w/v), upon which it is reported to rapidly precipitate as the sparingly soluble monohydrate (2-3% w/v). It is reported that below 85% relative humidity, famciclovir is not hydroscopic. Its partition coefficients are reported to be: octanol/water (pH 4.8) P=1.09 and octanol/phosphate buffer (pH 7.4) P=2.08.

U.S. Pat. No. 5,246,937 discloses that famciclovir may be produced by the hydrogenolysis of a compound of formula I (acetic acid 2-acetoxymethyl-4-(5-amino-7-chloro-imidazo[4,5-b]pyridin-3-yl)-butyl ester, or 2-[2-(2-amino-4-chloro-9H-purin-9-yl)ethyl]-1,3-propane diacetate) (Cl-FMC) in a palladium on charcoal (Pd/C) catalyst in methanol containing ammonium formate:

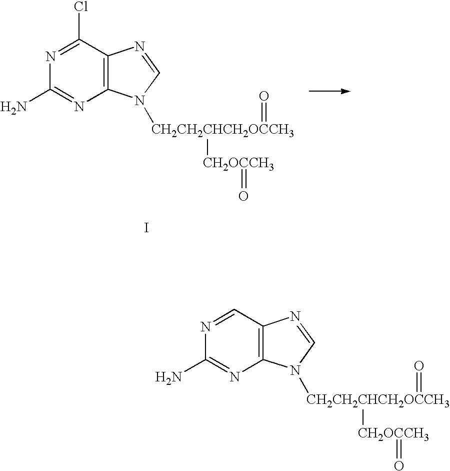

However, this process leads to high levels of two impurities:

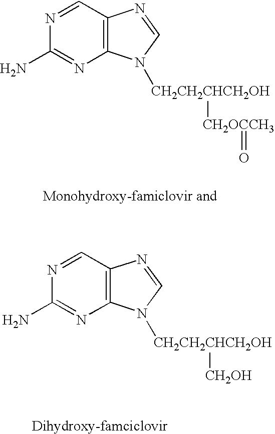

Thus, there remains a need for a process for producing famciclovir with low levels of undesirable by-products.

### SUMMARY OF THE INVENTION

The present invention provides processes for preparing famciclovir. In a first embodiment, the process comprises reacting a compound of formula I (acetic acid 2-acetoxymethyl-4-(5-amino-7-chloro-imidazo[4,5-b]pyridin-3-yl)-butyl ester) in the presence of a catalyst in a C₁-C₆ alkyl acetate and ammonium formate. In a second embodiment, the process comprises reacting a compound of formula I (acetic acid 2-acetoxymethyl-4-(5-amino-7-chloro-imidazo[4,5-b]pyridin-3-yl)-butyl ester) in the presence of a catalyst in a mixture of a C₁-C₆ alkyl acetate, a C₁-C₄ alcohol and ammonium formate.

The present invention provides famciclovir containing less than about 0.1% monohydroxy-famciclovir, preferably less than about 0.03%, by area percent HPLC. The present invention further provides famciclovir containing less than about 0.02% dihydroxy-famciclovir by area percent HPLC.

The present invention further provides a stable famciclovir that does not increase its monohydroxy impurity content upon storage for at least 6 months at either 25° C., 40° C. or 55° C. and 75% relative humidity.

### DETAILED DESCRIPTION OF THE INVENTION

Definitions: FMC refers to famciclovir; Cl-FMC refers to (acetic acid 2-acetoxymethyl-4-(5-amino-7-chloro-imidazo[4,5-b]pyridin-3-yl)-butyl ester (a compound of formula 1); MH-FMC refers to monohydroxy-famciclovir; DH-FMC refers to dihydroxy-famciclovir; Pd/C refers to palladium catalyst; RRT refers to relative retention time. Unless otherwise specified, % refers to % by area percent HPLC.

The present invention provides novel processes for the synthesis of famciclovir. These processes are advantageous as they yield famciclovir containing less than about 3% monohydroxy-famciclovir, without additional work-up steps.

In a first embodiment, the invention provides a process for making famciclovir (FMC), which comprises reacting a compound of formula I (acetic acid 2-acetoxymethyl-4-(5-amino-7-chloro-imidazo[4,5-b]pyridin-3-yl)-butyl ester (Cl-FMC)) in the presence of a catalyst in a C₁-C₆ alkyl acetate and ammonium formate. The alkyl acetate is preferably methyl, ethyl, propyl or butyl acetate. Most preferably, the alkyl acetate is ethyl acetate. When the alkyl acetate used is ethyl acetate, the reaction temperature is preferably between about 50-70° C. The catalyst is selected from the group consisting of palladium on charcoal and platinum. Preferably the catalyst is palladium on charcoal. The catalyst can be wet or dry. Preferably, the catalyst is wet. Most preferably the catalyst is about 50% (w/w) wet. The catalyst is preferably used in an amount of about 5-10% (w/w), most preferably about 10% (w/w).

In a second embodiment, the process comprises reacting a compound of formula I (acetic acid 2-acetoxymethyl-4-(5-amino-7-chloro-imidazo[4,5-b]pyridin-3-yl)-butyl ester (Cl-FMC)) in the presence of a catalyst in a mixture of a C₁-C₆ alkyl acetate, a C₁-C₄ alcohol and ammonium formate. The alkyl acetate is preferably methyl, ethyl, propyl or butyl acetate. The C₁-C₄ alcohol is preferably selected from the group consisting of methanol, ethanol, propanol and isopropanol. The ratio of alkyl acetate to alcohol is preferably from about 1:9 to about 9:1. Most preferably, the solvent is a mixture of methyl acetate to methanol (about 9:1). The catalyst is selected from the group consisting of palladium on charcoal and platinum, preferably the catalyst is palladium on charcoal. The palladium on charcoal catalyst can be wet or dry. Preferably, the catalyst is wet and is used in an amount of about 5-10% (w/w). Most preferably the catalyst is about 50% (w/w) wet, and is used in an amount of about 10% (w/w). These process yield famciclovir containing a MH-FMC level of less than about 3% by area percent HPLC. These processes also yield famciclovir containing a level of DH-FMC of less than about 0.02% by area percent HPLC.

In a third embodiment, the invention provides famciclovir containing less than about 0.1% monohydroxy-famciclovir, preferably less than about 0.03% by area percent HPLC. The famciclovir also contains less than about 0.02% dihydroxy-famciclovir by area percent HPLC.

In a fourth embodiment, the invention provides stable famciclovir, which does not increase its monohydroxy impurity level when stored at either 25° C., 40° C. or 55° C. and 75% relative humidity for at least 6 months.

The work-up of the product is simplified because the filtrate obtained after the filtration of the Pd/C contains mainly the product. The inorganic salts remain on the filter paper with the Pd/C. This separation allows the isolation of pure FMC in high yield and in excellent quality.

The reaction in MeOAc:MeOH (9:1; v/v) runs with either dry Pd/C or wet (50% H₂O; w/w) Pd/C. The MH-FMC level is not affected significantly by using dry or wet Pd/C.

The amount of Pd/C required to fully consume Cl-FMC is about 10% (w/w). However, about 5.5% (w/w) Pd/C is enough if the reaction time is extended to 8 hours. The level of the contaminants does not change significantly.

When the reaction was carried out in MeOH as the only solvent, at the end of the reaction, the level of MH-FMC was over about 3% by area percent HPLC.

In case there is a high level of impurities (i.e., Cl-FMC, MH-FMC and DH-FMC) it is advised to concentrate the filtrate (after the removal of Pd/C) to 25% its volume, then filter the solid. This solid will contain less of the impurities.

The purity of famciclovir was analyzed by HPLC under the following conditions:

| | |
|---|---|
| Pumping system: | HP model 1050 |
| Detector: | HP model 1100, l = 309 nm |
| Flow: | 1.2 ml/min |
| Injection: | 20 ml |
| Column: | ACE, C-18, 250 * 4.6 mm * 5 mm |
| Solvents: | A: H₂O (AmAc 0.1M) |
| | B: CH₃CN |

| Time | Solvent A | Solvent B |
|---|---|---|
| 0 | 85 | 15 |
| 5 | 85 | 15 |
| 10 | 50 | 50 |
| 20 | 50 | 50 |
| 25 | 85 | 15 |

Having thus described the various aspects of the present invention, the following examples are provided to illustrate specific embodiments of the present invention. They are not intended to be limiting in any way.

### EXAMPLES

Note: The composition of reaction mixture and solids are given as area % HPLC.

### Example 1

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added dry 11.4% Pd/C (2.3 g), MeOAc (200 ml), MeOH (20 ml), Cl-FMC (20 g; 55.8 mmol), and ammonium formate (10.5 g; 97% pure). The reaction mixture was heated at 40° C. Complete conversion of Cl-FMC was observed within 1 hr and 40 min. Under these conditions two hydrolysis by-products, mono-hydroxy FMC (MH-FMC) and di-hydroxy FMC (DH-FMC) were formed, 0.9% and 0.64%, respectively. The reaction mixture was left to stir for a total of 4 hours at 40° C. This did not have a significant effect on the level of the by-products that remained steady, i.e., the MH-FMC and DH-FMC level was 1.05% and 0.72%, respectively. The reaction was cooled to room temperature, then filtered. This solid retained 0.9% MH-FMC and only 0.6% of the DH-FMC.

### Example 2

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added dry 7% Pd/C (3.48 g), MeOAc (500 ml), MeOH (50 ml), Cl-FMC (50.1 g; 49.6 mmol), and ammonium formate (27.18 g; 97% pure). The reaction mixture was heated at 40° C. for 6 hours and 10 min. At this stage 98.37% FMC, 0.82% MH-FMC and 0.63% DH-FMC and 0.18% Cl-FMC; were detected. Upon cooling the reaction mixture and filtering the black solid, the filtrate was evaporated to dryness leaving 43.77 g solid (almost the entire expected amount of FMC; assay 97.3% FMC). The composition of the solid was left unchanged from the above quotation. The solid was partitioned in EtOAc (365 ml) and water (150 ml). The organic phase was washed with H₂O (45 ml), and kept aside. The combined aqueous phases were washed with EtOAc (3×40 ml). The combined EtOAc extracts were washed with H₂O (40 ml), and kept aside. The aqueous phases were extracted again with EtOAc (2×40 ml). The EtOAc extracts were washed with H₂O (10 ml), and kept aside. The three organic phases were combined, dried with MgSO₄ and evaporated to dryness leaving 34.7 g white solid (˜76.8% yield). The solid was crystallized in BuOH (100 ml; 63° C.) giving 28.9 g pure FMC (64% yield). Less then 0.1% impurities (each) were detected in the crystals.

### Example 3

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added dry 5.38% Pd/C (1.07 g), MeOAc (200 ml), MeOH (20 ml), Cl-FMC (20.04 g; 55.85 mmol), and ammonium formate (10.48 g; 97% pure). The reaction mixture was heated at 40° C. for 6 hours. The reaction mixture composition was 96.7% FMC, 0.86% DH-FMC, 1.07% of MH-FMC and 1.27% Cl-FMC. The reaction mixture was cooled to room temperature, filtered and the filtrate was evaporated to dryness leaving 17.94 g of white solid (assay 94.8% FMC).

### Example 4

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added 5.46% Pd/C wet (2.27 g; 50% H₂O), MeOAc (220 ml), Cl-FMC (21 g; 58.5 mmol), and ammonium formate (10.66 g; 97% pure). The reaction mixture was heated at 40° C. for 4.5 hours. The reaction mixture composition was 74.47% FMC, 0.64% DH-FMC, 0.74% of MH-FMC and 35% Cl-FMC. MeOH was added (20 ml) and the reaction mixture was left for 1 more hour at 40° C., then cooled to room temperature and stirred overnight. The reaction proceeded further and the composition of the reaction mixture was 89.6% FMC, 0.73% DH-FMC, 0.9% of MH-FMC and 8.8% Cl-FMC. Heating was continued 135 min. more at 40° C. and the composition of the reaction mixture was 93.05% FMC, 0.81% DH-FMC, 1% of MH-FMC and 5% Cl-FMC. The reaction mixture was filtered and the filtrate was concentrated to 40.3 g of slurry. The solid was filtered and washed to give 13.64 g solid composed of 97.53% FMC, 0.34% DH-FMC, 0.69% of MH-FMC and 1.44% Cl-FMC (assay 96.5% FMC).

### Example 5

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added wet 5.7% Pd/C (2.27 g; 50% H₂O), MeOAc (110 ml), MeOH (110 ml), Cl-FMC (20 g; 55.74 mmol), and ammonium formate (10.54 g; 97% pure). The reaction mixture was heated at 40° C. for 4 hours. The reaction mixture composition was 96.3% FMC, 1.06% DH-FMC, 2.64% of MH-FMC. No Cl-FMC was detected. The reaction mixture was cooled, filtered. The filtrate was concentrated to 53.5 g slurry. The white solid was filtered and washed with hexane, leaving 19.24 g solid (assay 87.5% FMC; expected amount 17.91 g).

### Example 6 (Comparative)

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added wet 5.56% Pd/C (4.4 g; 50% H₂O), MeOH (440 ml), Cl-FMC (40 g; 111.5 mmol), and ammonium formate (21.96 g; 97% pure). The reaction mixture was heated at 40° C. for 4.5% hours. The reaction mixture composition was 95.8% FMC, 0.55% DH-FMC, 3.3% of MH-FMC. No Cl-FMC was detected. The reaction mixture was cooled and filtered.

### Example 7

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added dry 5.4% Pd/C (1.07 g), EtOAc (220 ml), MeOH (20 ml); Cl-FMC (20 g; 55.8 mmol), and ammonium formate (10.5 g; 97% pure). The reaction mixture was heated at 40° C. for 5 hours 10 min. The reaction mixture composition was 50.57% FMC, 0.54% DH-FMC, 0.53% of MH-FMC and 48.36% Cl-FMC. MeOH was added (20 ml) and the reaction mixture continued at 40° C. for 80 min. more. Leaving the composition unchanged. The reaction mixture was left to stir overnight at room temperature. While the Cl-FMC was consumed slightly more (44.1%) the MH-FMC and DH-FMC level remained steady. After heating to 40° C. 3 hours more showed a progress of the reaction to 76.7% FMC, 0.67% DH-FMC, 0.93% MH-FMC and 21.53% Cl-FMC. More MeOH was added (40 ml) and the reaction was continued 2.5 hours. The composition of the reaction mixture was 96.14% FMC, 0.79% DH-FMC, 1.3% of MH-FMC and 1.55% Cl-FMC. After cooling to room temperature, the reaction mixture was filtered and the filtrate was evaporated to dryness leaving 17.45 g solid composed of 97.48% FMC, 0.64% DH-FMC, 1.14% of MH-FMC and 0.72% Cl-FMC (assay 97.1% FMC).

### Example 8

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added wet 10% Pd/C (3.92 g; 50% H₂O), i-BuOAc (155 ml), MeOH (65 ml); Cl-FMC (20 g; 55.8 mmol), and ammonium formate (11 g; 97% pure). The reaction mixture was heated at 40° C. After 75 min. all the Cl-FMC was consumed. At this level, 0.71% DH-FMC and 1.85% MH-FMC were formed.

### Example 9

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer, a reflux condenser and a thermocouple, under an inert atmosphere (N₂), was added wet 10% Pd/C (12 g, 50% H₂O), EtOAc (660 ml), Cl-FMC (60 g; 168.6 mmol), and ammonium formate (32.8 g; 504.5 mmol; 97% pure). The reaction mixture was heated at 50° C. After 5 hours, all the Cl-FMC was consumed, and the composition of the reaction mixture showed 99.6% FMC, 0.1% of MH-FMC and traces of the DH-FMC. The reaction mixture was filtered at 50° C. and the filtrate was evaporated to dryness, leaving 51.5 g of solid (95% of the 54.2 g expected). The solid was crystallized from n-BuOH (61° C.; 155 ml). After cooling in ice-water bath, filtration and drying (6 hrs; 60° C.) 48.6 g of pure FMC (89.7% yield) was obtained (99.8% FMC; MH-FMC level was less than 0.1% HPLC area %).

### Example 10

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipment with a mechanical stirrer, a reflux condenser and a thermocouple, under an inert atmosphere (N₂), was added wet 10% Pd/C (4 g, 50% H₂O), EtOAc (220 ml), Cl-FMC (20 g; 56.1 mmol) and ammonium formate (4.37 g; 67.28 mmol; 20% excess). The reaction was completed after 2 hours, as all the C1-FMC was consumed. The reaction mixture was filtered at 50° C. and the filtrate was evaporated to dryness, leaving 16.4 g of solid (90.9% of the 18 g expected).

### Example 11

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Cl-FMC (145 g), 10% Pd/C (28.92 g), and ammonium formate (31.7 g) were dissolved in EtOAc (1,450 ml) at 70° C. After 5 hours hot filtration was performed, the solution was concentrated by distillation of EtOAc (vacuum, 41° C.). After complete dissolution of the precipitated solid at 60° C., the solution was cooled for 1 hour to 5° C. and left overnight before separating the resulting product. Reaction yield −75% (based on Cl-FMC).

### Example 12

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer and a reflux condenser, under an inert atmosphere (N₂), was added wet 10% Pd/C (18.9 g, 52% H₂O), EtOAc (423 ml) and Cl-FMC (90 g; 252.9 mmol). The reaction mixture was heated to 70° C. Ammonium formate (19.7 g; 312.4 mmol) was added in 11 portions. The portions were added every 20 min. After 4 hours, all the Cl-FMC was consumed. The reaction mixture was diluted to 720 ml and filtered at 40° C. A charcoal (4.5 g) was added to the filtrate and the mixture was stirred for 30 min. Then the charcoal was filtered out and washed (90 ml) EtOAc. The wash was added to the filtrate. The filtrate was distillated back to 423 ml of EtOAc. Precipitation occurred during the distillation. The mixture was heated until a clear solution was obtained. Then the solution was cooled (4 hrs; 101° C.) and precipitation occurred during the cooling process. After 12 hrs of stirring, the material was filtered out and was washed with water, and a wet famciclovir was obtained. The material was dried 3 hr at 45° C. and 3 hr at 65° C. FMC (62% yield) was obtained (MH-FMC level was 0.03% HPLC).

### Example 13

### Preparation of Acetic acid 2-acetoxymethyl-4-(2-amino-purin-9-yl)-butyl ester (FMC) from Acetic acid 2-acetoxymethyl-4-(5-amino-7-chloro-imidazo[4,5-b]pyridin-3-yl)-butyl ester (Cl-FMC)

Into a jacketed reactor equipped with a mechanical stirrer and a reflux condenser, under an inert atmosphere (N₂), was added wet 10% Pd/C (68.7 g, 52% H₂O), EtOAc (1550 ml) and Cl-FMC (330 g; 927.5 mmol). The reaction mixture was heated to 50° C. Ammonium formate (71.2 g; 1130.3 mmol) was added in 11 portions. The portions were added every 20 min. After 5 hours, all the Cl-FMC was consumed. The reaction mixture was diluted to 2640 ml and filtered at 50° C. A charcoal (16.5 g) was added to the filtrate and the mixture was stirred for 30 min. Then the charcoal was filtered out and washed (330 ml) of EtOAc. The wash was added to the filtrate. The filtrate was distillated back to 1,550 ml of EOtAc. The mixture was heated until a clear solution obtained. Then the solution was cooled (5.5 hrs; −101° C.) and precipitation occurred during the cooling process. After 12 hr of stirring, the material was filtered out and was washed with water, and a wet famciclovir was obtained. The material was dried 3 hr at 45° C. and 3 hr at 65° C. FMC (69.8% yield) was obtained (MH-FMC level was 0.06% HPLC).

### Example 14

### Stability studies of the prepared Acetic acid 2-acetoxymethyl-4-(2-amino-purin-9-yl)-butyl ester (FMC)

Famciclovir prepared in Example 13 (above) was stored under various specified conditions. The stability of the famciclovir was evaluated at various times (i.e., 1-6 months) after storage under 75% relative humidity and various temperature conditions (i.e., 25° C., 40° C., and 55° C.). Purity of famciclovir and its by-products were measured by HPLC, the procedure of which has been detailed (see above). The result of famciclovir stability is shown below.

| | | | RRT (%) | | | | | | | | | | |
|---|---|---|---|---|---|---|---|---|---|---|---|---|---|
| FMC | | | MH-FMC | | | | | | | | | | |
| Batch | Time | T° C. | 0.51 | 0.80 | 0.87 | 0.89 | 1.10 | 1.27 | 1.35 | 1.74 | Any, % | Total, % | Appear. |
| 1 | start | | | | | | | | | | 0.00 | 0.00 | |
| | 1 month | 25° C. | | | | | | | | | 0.00 | 0.00 | |
| | 2 month | | | | | | | | | | 0.00 | 0.00 | |
| | 3 months | | | | | | | | | | 0.00 | 0.00 | |
| | 5 months | | 0.06 | 0.01 | 0.02 | 0.01 | 0.01 | 0.01 | 0.01 | 0.01 | 0.06 | 0.14 | White |
| | 6 months | | 0.06 | 0.01 | 0.01 | | | 0.01 | | | 0.06 | 0.09 | |
| | 1 month | 40° C. | | | | | | | | | 0.00 | 0.00 | |
| | 2 month | | 0.06 | <0.01 | <0.01 | 0.01 | <0.01 | <0.01 | <0.01 | <0.01 | 0.06 | 0.07 | White |
| | 3 months | | 0.06 | <0.01 | <0.01 | 0.01 | <0.01 | <0.01 | <0.01 | <0.01 | 0.06 | 0.07 | White |
| | 6 months | | 0.06 | 0.01 | 0.01 | | | 0.01 | | | 0.06 | 0.09 | |
| | 1 month | 55° C. | | | | | | | | | 0.00 | 0.00 | |
| | 2 month | | 0.07 | <0.01 | <0.01 | 0.01 | <0.01 | <0.01 | <0.01 | <0.01 | 0.07 | 0.08 | White |
| | 3 months | | 0.06 | <0.01 | <0.01 | 0.01 | <0.01 | <0.01 | <0.01 | <0.01 | 0.06 | 0.07 | White |
| | 6 months | | 0.06 | 0.01 | 0.01 | | | 0.01 | | | 0.06 | 0.09 | |

RRT (%) refers to % of relative retention time.

The disclosures of the cited publications are incorporated herein in their entireties by reference. It is to be understood, however, that the scope of the present invention is not to be limited to the specific embodiments described above. The invention may be practiced other than as particularly described and still be within the scope of the accompanying claims.

What is claimed is:

1. A process of preparing famciclovir comprising the steps of:
a) reacting acetic acid 2-acetoxymethyl-4-(5-amino-7-chloro-imidazol [4,5-b]pyridin-3-butyl ester in the presence of a catalyst in a C₁-C₆ alkyl acetate and ammonium formate; and
b) isolating famciclovir.

2. The process of claim 1, wherein the isolated famciclovir contains less than about 3% by area percent HPLC monohydroxy-famciclovir.

3. The process of claim 2, wherein the isolated famciclovir contains less than about 0.02% by area percent HPLC dihydroxy-famciclovir.

4. The process of claim 1, wherein the alkyl acetate is ethyl acetate.

5. The process of claim 4, wherein the isolated famciclovir contains less than about 0.03% by area percent HPLC monohydroxy-famciclovir.

6. The process of claim 4, wherein step (a) is performed between about 50° C. to about 70° C.

7. The process of claim 1, wherein the catalyst is selected from the group consisting of palladium on charcoal and platinum.

8. The process of claim 7, wherein said catalyst is palladium on charcoal.

9. The process of claim 8, wherein said palladium on charcoal catalyst is either dry or wet.

10. The process of claim 9, wherein said palladium on charcoal catalyst is about 50% (w/w) wet.

11. The process of claim 8, wherein said palladium on charcoal catalyst is used in an amount of from about 5% (w/w) to about 10% (w/w).

12. The process of claim 11, wherein said palladium on charcoal catalyst is used in an amount of about 10% (w/w).

13. The process of any one of the preceding claims, wherein the reaction mixture of step (a) further comprises a C₁-C₄ alcohol.

14. The process of claim 13, wherein the C₁-C₄ alcohol is selected from the group consisting of methanol, ethanol, propanol, and isopropanol.

15. The process of claim 13, wherein the mol/mol ratio of alkyl acetate to C₁-C₄ alcohol is between about 1:9 to about 9:1.

16. The process of claim 13, wherein the reaction mixture contains methyl acetate and methanol in a mol/mol ratio of about 9:1.

17. A process for preparing famciclovir comprising the steps of:
a) reacting acetic acid 2-acetoxymethyl-4-(5-amino-7-chloro-imidazol[4,5-b]pyridin-3-yl)-butyl ester in the presence of a palladium on charcoal catalyst in a C₁-C₆ alkyl acetate and ammonium formate;
b) concentrating the reaction mixture; and
c) isolating famciclovir.

18. The process of claim 17, wherein the reaction mixture of step (a) further comprises a C₁-C₄ alcohol.

19. The process of claim 17, wherein prior to step (b), the palladium on charcoal catalyst is filtered out.

20. Famciclovir prepared according to the process of claim 4.

21. Famciclovir containing less than about 0.03% by area percent HPLC monohydroxy-famciclovir.

22. The famciclovir of claim 21, containing less than about 0.02% by area percent HPLC dihydroxy-famciclovir.

23. Stable famciclovir that does not increase its monohydroxy impurity content to greater than 0.1% by area percent HPLC upon storage for at least 6 months at 25° C. and 75% relative humidity.

24. Stable famciclovir that does not increase its monohydroxy impurity content to greater than 0.1% by area percent HPLC upon storage for at least 6 months at 40° C. at 75% relative humidity.

25. Stable famciclovir that does not increase its monohydroxy impurity content to greater than 0.1% by area percent HPLC upon storage for at least 6 months at 55° C. at 75% relative humidity.

26. Stable famciclovir that does not increase its monohydroxy impurity content to greater than 0.06% by area percent HPLC upon storage for at least 6 months at 25° C. and 75% relative humidity.

27. Stable famciclovir that does not increase its monohydroxy impurity content to greater than 0.06% by area percent HPLC upon storage for at least 6 months at 40° C. at 75% relative humidity.

28. Stable famciclovir that does not increase its monohydroxy impurity content to greater than 0.06% by area percent HPLC upon storage for at least 6 months at 55° C. at 75% relative humidity.
